(19) United States
(12) Patent Application Publication
(10) Pub. No.: US 2025/0376703 A1
(43) Pub. Date: Dec. 11, 2025

(54) METHOD FOR PROCESSING LIGNOCELLULOSIC BIOMASS

(71) Applicant: IFP ENERGIES NOUVELLES, Rueil-Malmaison (FR)

(72) Inventors: Matthieu ARAGONES, RUEIL-MALMAISON CEDEX (FR); Caroline AYMARD, RUEIL-MALMAISON CEDEX (FR); Sophie COUDERC, RUEIL-MALMAISON CEDEX (FR); Gilles FERSCHNEIDER, RUEIL-MALMAISON CEDEX (FR)

(73) Assignee: IFP ENERGIES NOUVELLES, Rueil-Malmaison (FR)

(21) Appl. No.: 18/875,054

(22) PCT Filed: Jun. 6, 2023
(86) PCT No.: PCT/EP2023/065093

§ 371 Date: Dec. 13, 2024

(30) Foreign Application Priority Data
Jun. 15, 2022 (FR) FR2205835

(51) Int. Cl. C12P 7/10

(52) U.S. Cl. CPC C12P 7/10; C12P 2201/00

(57) ABSTRACT

The invention relates to a process for treating a lignocellulosic biomass, with the use of at least one reactor (9, 14) for treating said biomass, said reactor (9, 14) being equipped with a feed device (6, 11) which is equipped with a biomass inlet and a biomass outlet, said biomass outlet being in fluidic connection with an inlet of the reactor, such that

a residue (8;13) is extracted from the biomass, while it is passing through the feed device (6;11) toward the reactor (9;14), via an extraction outlet provided in said device, said residue being a mixture of solid and liquid,

said residue is separated into a solid residue (32; 42) and a liquid residue (31; 41),

at least one portion of the solid residue (32; 42) is reintroduced into the feed device, or into one of said feed devices.

### TECHNICAL FIELD

The invention relates to a process for treating lignocellulosic biomass for producing “second-generation” (2G) sugary liquors. These sugary liquors may be used to produce other products via a biochemical pathway (for example alcohols such as ethanol, butanol or other molecules, for example solvents such as acetone, etc.). This process generally comprises three steps, namely liquor preparation, impregnation of the biomass with this liquor, and pretreatment of the impregnated biomass, for example by cooking, optionally coupled with steam explosion.

### PRIOR ART

Lignocellulosic biomass represents one of the most abundant renewable resources on Earth. The substrates considered are very varied, they relate both to ligneous substrates such as various woods (hardwoods and softwoods), coproducts derived from agriculture (wheat straw, corn cobs, etc.) or from other agrifood, paper, etc. industries.

The process for the biochemical conversion of the lignocellulosic biomass into 2G sugary liquors notably comprises a pretreatment step and a step of enzymatic hydrolysis with an enzyme cocktail. These processes also usually include an impregnation step before the pretreatment. The sugary liquors resulting from the hydrolysis are then treated, for example by fermentation, and the process also comprises separation steps and/or a step of purification of the final product.

Lignocellulosic biomass is composed of three main polymers: cellulose (35% to 50% by weight), which is a polysaccharide essentially consisting of hexoses; hemicellulose (20% to 30% by weight), which is a polysaccharide usually consisting essentially of pentoses; and lignin (15% to 25% by weight), which is a polymer of complex structure and of high molecular weight, composed of aromatic alcohols connected via ether bonds. These various molecules are responsible for the intrinsic properties of plant walls and are organized in a complex entanglement.

Among the three base polymers that make up lignocellulosic biomass, cellulose and hemicellulose are the ones that enable the production of 2G sugary liquors.

Usually, hemicellulose is predominantly broken down into sugar during the pretreatment, and cellulose is converted into glucose by enzymatic hydrolysis. However, crude cellulose remains difficult for enzymes to access, hence the need for a pretreatment. This pretreatment makes it possible to modify the physicochemical properties of the lignocellulosic biomass so as to improve the accessibility of the cellulose to enzymes and its reactivity to enzymatic hydrolysis.

There are many advantageous technologies for performing this pretreatment, which will be combined below under the generic term “cooking”: acidic cooking, alkaline cooking, cooking by auto-hydrolysis, steam explosion, and “organosolv pulping” processes. The latter process concerns pretreatment in the presence of one or more organic solvents and generally water. The solvent may be an alcohol (ethanol), an acid such as acetic acid or formic acid, or else acetone. “Organosolv pulping” processes lead to at least partial dissolution of the lignin and partial dissolution of the hemicelluloses. There are thus two outlet streams: the pretreated substrate with residual cellulose, hemicellulose and lignin, and the solvent phase which contains the dissolved lignin and a portion of the hemicelluloses. There is generally a step of regeneration of the solvent, which makes it possible to extract a lignin stream. Certain “organosolv pulping” treatments (notably with ethanol) are coupled with the addition of a strong acid (such as H₂SO₄). It may also be envisaged to place the biomass in contact with the solvent via an impregnation reactor before the cooking phase or to place the biomass in contact with the acid catalyst before performing “organosolv pulping” cooking.

Various configurations are reported, for example, in the publication “Production of bioethanol from lignocellulosic materials via the biochemical pathway: A review”, M. Balat, Energy Conversion and Management 52 (2011) 858-875, or in the publication “Bioethanol production from agricultural wastes: An overview”, N. Sarkar, S. Kumar Ghosh, S. Bannerjee, K. Aikat, Renewable Energy 37 (2012) 19-27.

One of the most effective pretreatments is steam explosion, notably under acidic conditions, which enables almost complete hydrolysis of hemicellulose and a significant improvement in the accessibility and reactivity of cellulose to enzymes. This pretreatment may be preceded by other treatment(s).

All these pretreatments are applied to biomasses which are initially in solid form: the aim of the pretreatment is to destructure them.

Patents U.S. Pat. No. 8,057,639 and U.S. Pat. No. 8,512,512 propose a process comprising a first step of hydrolysis of hemicellulose to C5 sugars under mild conditions which thus protect them from degradation. This step is performed in a first reactor at a pressure of 1.5 bar (0.15 MPa) or more, by injection of steam, at a temperature of 110° C. or more, and optionally in the presence of a weak acid.

After this step, washing is performed in order to extract and recover the sugar liquors obtained from hemicellulose (generally liquors of C5 sugars and C6 sugars, the relative proportion of which depends, in particular, on the nature of the biomass) before sending the remaining biomass, enriched in cellulose and lignin, to a second step (second reactor) where the steam explosion takes place. This second reactor operates at a higher pressure than the first reactor with an injection of high-pressure steam which causes a sudden expansion of the biomass (steam explosion).

When a treatment requires a pressure step (impregnation, pretreatment of cooking type or the like), it is necessary to make use of solid biomass introduction means that are compatible with these pressure steps. This is the case, for example, of compression screws, one embodiment of which is described in patent U.S. Pat. No. 4,599,138.

Patent FR 3 075 203 describes a process involving impregnation of biomass with an acidic liquor, followed by cooking and steam explosion of the impregnated biomass, with adjustment of the acidity of the acidic liquor and recycling thereof. Patent FR 3 075 201 also describes a process for pretreating biomass by acid impregnation followed by steam explosion, along with washing of the reactor feed means and recycling of the washing water into the process.

The aim of the invention is then to improve the lignocellulosic biomass treatment. The aim of the invention is notably to improve the introduction of biomass into a reactor performing one or more of the biomass treatment steps.

The aim of the invention is more particularly to improve the steps of impregnation and/or steam explosion cooking of the biomass as described in the abovementioned prior documents. An aim of the invention is also to make the treatment process, and notably these two steps, more efficient, in terms of energy and/or treatment fluid consumption and/or biomass conversion efficiency.

### SUMMARY OF THE INVENTION

The invention relates firstly to a process for treating a lignocellulosic biomass comprising a solids content of at most 90% by weight, said process comprising the use of at least one reactor for treating said biomass, said reactor being equipped with a biomass feed device which is equipped with a biomass inlet and a biomass outlet, said biomass outlet being in fluidic connection with an inlet of the reactor. The process is characterized by the fact
that a residue is extracted from the biomass, while it is passing through the feed device toward the reactor, via an extraction outlet provided in said device, said residue, referred to as solid-liquid residue, being a mixture of solid and liquid,
and that said solid-liquid residue is separated into a solid residue and a liquid residue,
and that at least one portion of the solid residue is reintroduced into the same feed device, or into one of said feed devices in the case of a plurality of reactors.

In the context of the invention, the expression “lignocellulosic biomass comprising a solids content of X %” means either biomass which naturally comprises a solids content of X % (“native” biomass) or biomass which has this content after one or more operations prior to the process according to the invention. And this solids content (acronym “SC”) denotes the solids content which is measured according to the standard ASTM E1756-08 (2015) “Standard Test Method for Determination of Total Solids in Biomass”.

In the context of the invention, a “solid-liquid residue” is understood to mean a liquid containing suspended solid (solid particles). A “solid residue” is understood to mean a residue which comprises at least 20% by weight of solid, (notably at least 30% or 40%, or even at least 50% by weight of solid depending on the separation method considered) and which has a solid consistency of sludge type or of acid-impregnated biomass fragments. This residue generally comprises between 20% and 50% by weight of solid.

A “liquid residue” is understood to mean a residue which comprises at least 50% by weight of liquid, notably at least 80% by weight of liquid and which has the consistency of a liquid free, or substantially free, of suspended solid particles.

The invention, as will be described in detail below, may advantageously be applied to any type of reactor used in a biomass conversion process, and more particularly to biomass impregnation reactors (when an impregnation is envisaged, whether this is by a liquor comprising a chemical compound such as an acid, a base or an oxidizing agent or by a water-based liquor, with autolysis of the biomass naturally releasing an acid, notably acetic acid) and/or to cooking or steam explosion heat treatment reactors.

The treatment of the lignocellulosic biomass in preceded by one or more pretreatment steps, such as, notably, at least one screening/cleaning operation in order to remove metal-type foreign elements (“removal of metals”) from the biomass, at least one optional mechanical grinding in order for the biomass to be reduced to particles having a size suitable for treatment in the/each of the treatment reactors, at least one washing, notably with water, of the biomass, etc. with an order and number of steps that are variable.

The invention did consist in proposing to extract from the biomass, before it enters the reactor, a residue which is partly solid and partly liquid, and to make optimal use thereof.

Specifically, it was already known to extract/recover a liquid (aqueous) residue/liquid washing water from the feed means of the reactors in question, in order to adjust the solids content of the biomass, in order to wash the equipment for feeding the reactor with biomass, etc. It was then a question of recovering this residue in order to reuse it “as is”, as aqueous liquid makeup in the process, by considering that it was simply a liquid, to be recovered in order to lower the water and/or chemical (acid of the impregnation liquor for example) consumption of the process.

However this extracted residue, although it might actually comprise a high proportion of liquid (water), also comprised solid material (even if only in the case of washing the feed equipment with water to prevent the fouling thereof): it is a liquid loaded with solid particles.

And it was pointed out by the inventors that, once separated from the rest of the extracted residue, this solid residue (these particles) had a composition that was very interesting and upgradable.

Thus, a solid residue, obtained from a solid-liquid residue extracted from a feed device of an impregnation reactor contains sugar polymers, lignin and has a formulation very similar to that of the biomass which is introduced into the feed device, which may be either native biomass, or biomass that has already undergone one or more treatments (mechanical treatment of grinding or metal removal type, or chemical treatment, of the type of adding water to adjust the solids content of the biomass before impregnation or other treatment).

A solid residue, obtained from a solid-liquid residue extracted from a feed device of a cooking reactor has a formulation which may differ from native biomass, notably if the latter has been impregnated beforehand with an acidic, basic or oxidizing liquor and/or has undergone other pretreatments: in the case of impregnation with a liquor, for example acid, the residue can contain traces of this liquor (for example it may contain an acid which is in the formulation of the liquor), it may also contain less hemicellulose, a portion of which has been able to be converted to sugars under the action of the impregnation liquor, it may contain less cellulose and lignin too. This residue may also contain fewer extractable species (monomeric sugars, ash, etc.) than the biomass since the impregnation acts on these soluble species.

The invention has then made good use of these observations in order to separately upgrade this solid residue in order to improve as best possible the performance of the process in its entirety, with quite a series of advantages:
by separating the solid-liquid residue, it is possible to recover a liquid residue free, or substantially free, of solid particles, which makes it possible to recycle this liquid more fully (the particles not disrupting/risking fouling the equipment recirculating the washing waters for example),
by recycling the separated solid residue, the efficiency of the process for the conversion of biomass to sugars is increased (by pretreatment generating sugars, notably C5 sugars, then enzymatic hydrolysis generating sugars of C6 sugar type) or of biomass to ethanol (by pretreatment, enzymatic hydrolysis and fermentation), since a biomass derivative which would otherwise be lost and which comprises sugars/components still capable of being converted to sugars is reincorporated into the biomass conversion process,
the liquid residue stripped of its solid particles can be used to reduce the water consumption of the process: the solid-liquid residue obtained from the feed device of a cooking reactor may thus contain up to 80% by weight of water, recovering this water is therefore very advantageous, since the process is water-intensive, and even the solid-liquid residue extracted from the feed device of the impregnation reactor may comprise a significant content of water, which is at least the water content of the native biomass, a content that varies depending on the nature of the biomass but which may rise to at least 30% by weight,
the liquid residue obtained from the solid-liquid residue extracted in the feed device of a cooking reactor and originating from an acid-impregnated biomass will contain a certain content of acid, and will therefore be able to be used as makeup for producing the impregnation liquor, and will thus lower the acid consumption of the process (obviously, same advantage of reduction of chemical consumption if the impregnation is carried out with a basic or oxidizing liquor). This liquid residue may also be used to correct for example the pH/amount of oxidising agent contained in the impregnation liquor and present in a residual manner in the biomass at various steps of its conversion process, outside of the preparation of the impregnation liquor itself.

According to the invention, the or at least one of the feed devices can be washed by circulation of a washing fluid between a washing inlet and a washing outlet of said feed device, the washing outlet preferably also being the extraction outlet for the solid-liquid residue.

In this case, the solid-liquid residue contains a high content of liquid (water) since it comprises the water contained in the biomass and the washing water.

According to the invention, at least one portion of the liquid residue can be reintroduced into the same feed device, or into one of said feed devices in the case of a plurality of reactors.

According to the invention, in one variant, the process comprises the use of several reactors for treating said biomass, at least one portion of the solid residue is reintroduced into one of the feed devices of said reactors, and at least one portion of the liquid residue is reintroduced into another of said feed devices.

In one variant, the solid residue obtained from a feed device of an impregnation reactor can be reintroduced at the inlet of the feed device, for example together with the biomass feedstock entering the device.

In another variant, the solid residue obtained from a feed device of a cooking reactor can be reintroduced at the inlet of the feed device, for example together with the biomass feedstock entering the device, which optionally has been impregnated beforehand.

It is also possible for the solid residue obtained from a feed device of an impregnation reactor to be sent to the feed device of the cooking reactor (ou vice versa), with less of a favorable impact, however, on the biomass conversion efficiency than in the two preceding variants.

Advantageously, at least one of the feed devices creates a pressure increase between the biomass inlet and the biomass outlet of said device, this pressure increase generating a compression of the biomass leading to the extraction of the solid-liquid residue.

One of the feed devices at least may thus be a feed screw, which is notably at least partly conical, comprising a cowling equipped with a cage equipped with openings which allow the extraction of the solid-liquid residue from the biomass and the circulation of a washing fluid. This compression screw (also referred to as “plug screw”), as is known, creates a hermetic plug of biomass in the downstream portion of the screw, which creates a compression on the biomass which is reflected by a pression difference between the biomass inlet and the biomass outlet of the screw, for example of at least 0.05 MPa, for example about 0.5 MPa. The compression applied to the biomass may thus lead to the expulsion of a portion of the liquid contained in the biomass, notably when the SC of the biomass is less than 80% before it enters the pressurized feed means.

According to the invention, the solid-liquid residue can be separated into a solid residue and a liquid residue by at least one separation device chosen from a centrifugation device, a draining or pressing device, or a screening unit.

The process according to the invention may comprise a step of impregnating the biomass with an impregnation liquor containing a chemical catalyst, said step being carried out by introducing the biomass into the or one of the reactors through its/their feed device.

The process according to the invention may comprise a step of treating the biomass by cooking or steam explosion, said step being carried out by introducing the biomass into the or one of the reactors through its/their feed device.

The process according to the invention may comprise a step of treating the biomass by enzymatic hydrolysis, said step being subsequent to the cooking or steam explosion thereof, in an enzymatic hydrolysis reactor equipped with its feed device. The feed device of this reactor is conventional, it may for example be an endless screw device (no compression of biomass at this stage generally).

The process according to the invention may comprise at least one step of treating the biomass by fermentation, said step being subsequent to or concomitant with the enzymatic hydrolysis step, in a fermentation reactor equipped with its feed device. The feed device of this fermentation reactor is conventional, it may for example be an endless screw device (no compression of biomass at this stage generally). It should be noted that the enzymatic hydrolysis and the fermentation may also be performed in the same reactor.

The process according to the invention may comprise at least one step of treating the biomass that aims to separate the solvents or alcohols, said step being subsequent to the fermentation step and being carried out in a separation reactor equipped with its feed device. As described in detail below, this reactor may be any piece of equipment, including, notably, at least one distillation column, the feed device thereof being any conventional device for this type of equipment.

According to one embodiment of the invention, at least one portion of the solid residue is reintroduced into the feed device of the impregnation reactor or into the feed device of the cooking reactor, and the liquid residue is preferably reused for the preparation of the impregnation liquor.

According to one embodiment of the invention, at least one portion of the solid residue is reintroduced into the feed device of the enzymatic hydrolysis reactor, and the liquid residue is preferably reused for the preparation of the impregnation liquor.

According to one embodiment of the invention, a residue can be extracted from the biomass, while it is passing through the feed device toward the impregnation reactor and/or toward the cooking reactor, via an extraction outlet provided in said device, said residue, referred to as solid-liquid residue, being a mixture of solid and liquid,
then said solid-liquid residue is separated into a solid residue and a liquid residue,
and at least one portion of the solid residue is reintroduced into the same feed device (of the impregnation or cooking reactor therefore) or into at least one of the feed devices of the enzymatic hydrolysis reactor or of the fermentation reactor or of the separation reactor.

In this configuration, a solid residue obtained from a biomass pretreatment (cooking with optional prior impregnation) is therefore reused in order to introduce it into a reactor downstream of the pretreatment reactor(s), which aim to convert the pretreated biomass (at the outlet of the cooking reactor) into sugars, (it is then referred to as hydrolyzate) then optionally into solvant or alcohol of ethanol type (it is then referred to as fermentation must from which the alcohol or solvent of interest is separated in a known manner).

According to one embodiment, the method according to the invention may comprise the following steps:
a) preparing an impregnation liquor containing a chemical catalyst intended for the impregnation of the biomass, catalyst chosen from an acid catalyst, a basic catalyst and an oxidizing catalyst, and preferably an acid catalyst, in a preparation zone,
b) introducing the biomass into an impregnation reactor by means of a first feed device, said first feed device being washed by circulation of a first washing fluid between a washing inlet and a washing outlet of said device (6),
c) introducing the liquor into the impregnation reactor via a first liquor (4) inlet of the reactor,
d) transferring the impregnated then drained biomass from an impregnation reactor outlet to an inlet of a cooking pretreatment reactor via at least one second feed device, said second feed device being washed by circulation of a second washing fluid between a washing inlet and a washing outlet of said feed device,
e) pretreating said biomass in said reactor by cooking or steam explosion,
f) extracting from the biomass a solid-liquid residue passing through at least one of the two feed devices, then separating said/each of the solid-liquid residues into a solid residue and a liquid residue with reintroduction of at least one portion of the solid residue as biomass makeup into at least one of the feed devices, and reintroduction of at least one portion of the liquid residue as washing fluid makeup into at least one of the feed devices or as makeup for the impregnation liquor in the liquor preparation zone or in the impregnation reactor.

The invention also relates to any plant implementing the process described above.

The plant according to the invention may thus comprise at least one biomass treatment reactor, said reactor being equipped with a biomass feed device which is equipped with a biomass inlet and a biomass outlet, said biomass outlet being in fluidic connection with an inlet of the reactor, with extraction from the biomass, while it is passing through the feed device toward the reactor, of a residue via an extraction outlet provided in said device, said residue, referred to as solid-liquid residue, being a mixture of solid and liquid, the plant also comprising
at least one device for separating said solid-liquid residue into a solid residue and a liquid residue, notably chosen from a centrifugation device, a draining or pressing device, or a screening unit,
means for reintroducing at least one portion of the solid residue into the same feed device, or into one of said feed devices in the case of a plurality of reactors,
optionally means for reintroducing at least one portion of the liquid residue into the plant, notably into the/one of the feed devices.

The means for reintroducing the liquid and/or solid residues are conventional, and may include any suitable hydraulic connection means (pipes, equipment of pump or filter type, valves).

Another subject of the invention is the use of the process or of the plant as described above for the treatment of lignocellulosic biomasses, of the type of wood, straw, agricultural residues, and all dedicated energy crops, in particular annual or perennial plants, such as miscanthus, in order to produce sugars, biofuels or biobased molecules.

### LIST OF FIGURES

FIG. 1 is a schematic representation of a plant for converting lignocellulosic biomass where the process according to the invention may be applied.

FIG. 2 is a block diagram of the process according to the prior art using the plant of FIG. 1.

FIG. 3 is a block diagram of a portion of the process according to the invention, modifying the process according to FIG. 2.

FIG. 4 is a block diagram illustrating a first variant of the process according to the invention.

FIG. 5 is a block diagram illustrating a second variant of the process according to the invention.

FIG. 6 is a block diagram illustrating a third variant of the process according to the invention.

FIG. 7 is a block diagram illustrating a fourth variant of the process according to the invention.

FIG. 8 is a block diagram illustrating a fifth variant of the process according to the invention.

FIG. 9 is a schematic representation of a variant according to the invention of the plant for converting lignocellulosic biomass described in FIG. 1.

It should be noted that the same references relate to the same stream or the same device from one figure to another.

### DESCRIPTION OF THE EMBODIMENTS

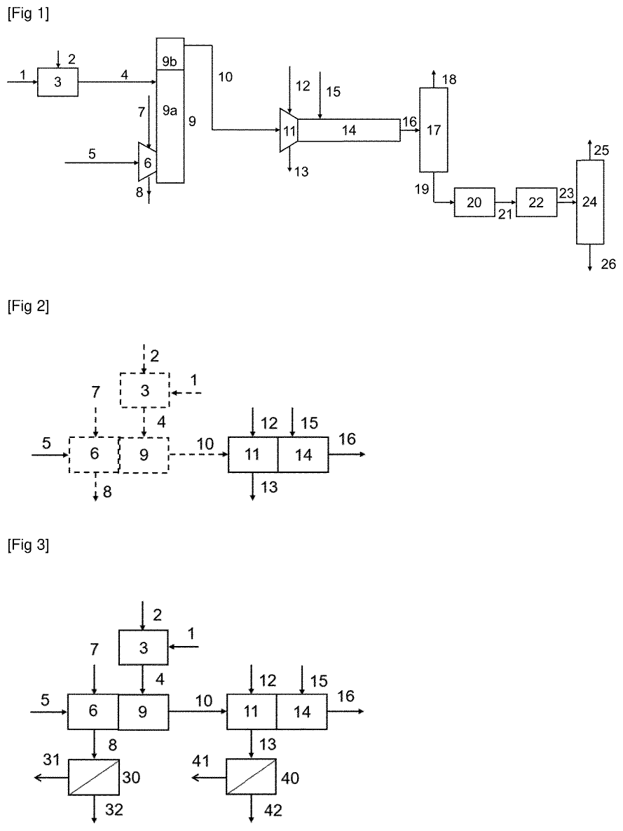

FIG. 1 very schematically represents a nonlimiting example of a plant for converting lignocellulosic biomass wherein the invention can be applied, which plant makes provision for a pretreatment of the biomass including an impregnation with an acidic liquor then a cooking/steam explosion, then an enzymatic hydrolysis followed by an alcoholic fermentation, in order to convert the biomass to ethanol.

The invention applies in an analogous manner to different plants, notably:
plants which target only the production of sugars, and which do not therefore make provision for alcoholic fermentation,
plants which make provision for a cooking pretreatment without a prior impregnation by a liquor (autolysis for example),
plants which make provision for a cooking pretreatment with a prior impregnation by a non-acidic liquor, for example a basic or oxidizing liquor.

Specifically, the invention is intended to be applied to any reactor, including, but not exclusively, impregnation and cooking reactors, which is used in a biomass conversion process.

FIG. 1 therefore represents a biomass conversion performed in the following manner: the ground biomass 5 (optionally impregnated with a catalytic liquor) is introduced into an impregnation reactor 9 by a pressurized feed means 6 which is a screw, also known as a plug screw feeder, the end portion of which is conical, which has a cowling with a drainage screen, a washing water inlet 7 and a washing water outlet 8. A hermetic plug of biomass is created in the downstream portion of the screw, which creates a compression on the biomass which is reflected by a pression difference between the biomass inlet and the biomass outlet of the screw of at least 0.05 MPa. The compression applied to the biomass may lead to the expulsion of a portion of the liquid contained in the biomass, notably when the SC of the biomass is less than 80% before it enters the pressurized feed means 6. The liquid thus extracted mixes with the washing water and is withdrawn with the used washing water 8.

The impregnation reactor is also supplied with acidic liquor 4 (water supplemented with sulfuric acid) originating from a liquor preparation tank 3, itself supplied with acid 2 and with water 1. The reactor comprises a biomass impregnation zone 9a, surmounted by a biomass drainage zone 9b.

The impregnated and drained biomass 10 leaves the reactor 9 in order to feed a steam explosion cooking reactor 14 via another feed screw 11 similar in its operation to the feed screw 6. In this screw, owing to the compression exerted on the biomass, a liquid 13 resulting from this pressing, also referred to as pressate, is recovered, which is composed of water and acid. The screw is washed, using a dedicated inlet, with a washing liquid 12 (water and/or a recycled liquid, as seen below), which is then discharged via the outlet through which the pressate 13 is also discharged.

The reactor 14 is also supplied with steam 15. On leaving the reactor 14, the biomass-steam mixture passes into a tool 17 for separating the biomass 19 and the steam 18. The biomass 19 is then treated in an enzymatic hydrolysis reactor 20, then once hydrolyzed to sugars, the hydrolyzed biomass 21 (also referred to as hydrolysis must) passes into an alcoholic fermentation reactor 22. The biomass fermented to alcohol 23, referred to as fermentation must, is then conveyed to one or more distillation columns to obtain concentrated alcohol 25 and crude vinasses 26.

This is only one example of a plant, which may also have numerous variants. Thus, the enzymatic hydrolysis and fermentation may be carried out together in the same reactor, it is then referred to as SSCF (simultaneous saccharification and co-fermentation).
1: Entry of water into the liquor preparation tank
2: Entry of acid into the liquor preparation tank
3: Liquor preparation tool (tank)
4: Acidic liquor to impregnation tool (reactor)
5: Ground biomass
6: Screw (plug-screw feeder) of the impregnation tool
7: Water for washing the plug-screw feeder of the impregnation tool
8: Washing liquid outlet of the plug-screw feeder 6 of the impregnation tool
9: Impregnation tool (reactor)
9a: Impregnation zone of the impregnation tank 9
9b: Drainage zone of the impregnation tank 9
10: Impregnated and drained biomass
11: Screw (plug-screw feeder) of the pretreatment tool
12: Water for washing the plug-screw feeder of the pretreatment tool
13: Pressate from the plug-screw feeder of the pretreatment tool
14: Pretreatment cooking tool (explosion reactor)
15: Injection of steam for the pretreatment
16: Pretreated biomass and steam
17: Tool (cyclone) for separating steam and pretreated biomass
18: Steam to condensation
19: Pretreated biomass
20: Enzymatic hydrolysis reactor
21: Hydrolysate containing sugars
22: Alcoholic (ethanolic) fermentation reactor
23: Fermentation wine containing ethanol (alcohol)
24: Ethanol recovery device, for example distillation column(s)
25: Concentrated alcohol
26: Residues (solid and liquid, as mixtures or separated depending on the arrangement of 24)

### The Process

Below is a more detailed description of the various steps of a biomass conversion process using such a plant and to which the invention may advantageously be applied: (this is one example, to which the invention is not limited).

### Step a) of Conditioning the Lignocellulosic Biomass

The treatment process comprises, in its first step, a step of conditioning the lignocellulosic biomass with at least one grinding so as to obtain biomass particles having a size of at most 300 mm. It is of course possible to carry out several successive grinding steps in order to reach the targeted particle size. In general, the ground biomass has a particle size (the largest size) of at most 300 mm, usually of at least 1 mm, and often between 2 and 200 mm. Any method known to those skilled in the art can be used to carry out this step. Usually, straw is ground with screens of from 5 to 100 mm. With regard to wood, it is generally chipped into parallelepipedal chips with a length of between 20 and 160 mm, a width of between 10 and 100 mm and a thickness of between 2 and 20 mm. The ground lignocellulosic biomass is conveyed to the next step by any means known to those skilled in the art, in particular a screw conveyor.

Step b) of Impregnation with an Acid Liquor

The treatment process according to the invention comprises a step b) of impregnating the lignocellulosic substrate with an acid liquor, so as to obtain an impregnated lignocellulosic substrate, the pH of which is between from 0.1 to 3. This step aims to prepare the lignocellulosic substrate for the pretreatment step.

The impregnation is carried out in an impregnation reactor at a temperature between 10° C. and 90° C., and preferably at atmospheric pressure. The residence time of the lignocellulosic substrate in the impregnation reactor is usually from 10 seconds to 180 minutes, preferably between 30 seconds and 60 minutes, and more preferentially still between 30 seconds and 15 minutes. Preferably, the impregnation step is performed in a single step.

The impregnation reactor or impregnator is equipped with one or more screws which transfer the lignocellulosic substrate from its inlet to the outlet opening. The impregnator is moreover equipped with one or more lines for conveying the acid liquor and also, if need be, one or more lines for withdrawing acid liquor. Said acid liquor inlet and outlet lines are generally installed so as to function by cocurrent or counter-current recycling.

The acid liquor is an aqueous solution of a strong acid, which is for example chosen from sulfuric acid, hydrochloric acid and nitric acid, for example at an acid content of between 0.5% and 4% by weight.

### Step c) of Solid/Liquid Separation on the Lignocellulosic Substrate Impregnated With Acid Liquor

In accordance with step c) of the treatment process according to the invention, the lignocellulosic substrate impregnated with acid liquor is subjected to a solid/liquid separation step in order to obtain a lignocellulosic substrate having a solids content of between 15% and 70% by weight and a used acid liquor. Preferably, the lignocellulosic substrate impregnated with acid liquor is first drained, in order to extract at least a portion of the free acid liquor, before being treated by solid/liquid separation.

The solid/liquid separation step may be carried out by any technique known to those skilled in the art, which may be for example decantation, centrifugation or pressing.

Preferably, pressing of the lignocellulosic substrate is carried out concomitantly with its transfer to pretreatment step d), when the latter carries out the steam explosion process which is described below. This method of carrying out step c) is for example performed by a screw referred to as a “plug screw feeder”, the operation of which has already been described above. The formation of a plug of pressed lignocellulosic substrate ensures the pressure-tightness of the steam explosion reactor, thus preventing dangerous escapes of steam. The screw conveyor is also provided with one or more lines for withdrawing the used liquor (referred to as pressate) separated during the pressing. The pressate can be recycled to the impregnation step b) and/or to the step of washing with the washing liquid 12 passing through the feed screw 11.

The wet biomass obtained at the end of the solid/liquid separation step c), which can be denoted by the term “washed and acidified lignocellulosic substrate” has a solids content preferably between 15% and 70% by weight, and more preferentially between 40% and 65% by weight.

### Step d) of Pretreating the Washed and Acidified Lignocellulosic Substrate

The washed and acidified lignocellulosic substrate, in accordance with step c) of the process, undergoes a pretreatment step d).

Cellulose (and optionally hemicelluloses) which are the targets of the enzymatic hydrolysis are not directly accessible to the enzymes. This is the reason why a pretreatment of the biomass is carried out before the enzymatic hydrolysis step. The pretreatment is notably directed toward modifying the physical and physicochemical properties of the cellulosic fraction, such as its degree of polymerization and its state of crystallinity.

Various types of pretreatment are known to those skilled in the art, they combine a chemical treatment and a heat treatment. Mention may in particular be made of acidic or basic cooking, the Organosolv process, treatments with ionic liquids and the steam explosion process. The preferred pretreatment process is steam explosion (or “SteamEx”) carried out in an acidic medium. This is a process in which the lignocellulosic substrate is rapidly brought to a high temperature by injecting pressurized steam. Stoppage of the treatment takes place by abrupt decompression.

The operating conditions of the steam explosion process are as follows:
steam is injected directly into the reactor;
the temperature of the reactor is generally between 150° C. and 220° C., preferably between 170° C. and 210° C.,
the pressure is between 5 and 25 bar absolute (0.5 and 2.5 MPa), more preferentially between 8 and 19 bar absolute (0.8 to 1.9 MPa),
the residence time before the expansion phase ranges from 10 seconds to 50 minutes and preferably between 3 minutes and 30 or 40 minutes.

The steam explosion may be performed in batch or continuous mode and the depressurization step which permits destructuring of the biomass may proceed in one or more steps.

At the end of the steam explosion pretreatment step, a pretreated lignocellulosic substrate with a high solids content, generally between 20% and 70% by weight and a vapor phase which is then condensed, are obtained.

In the context of the invention, the pretreated lignocellulosic substrate obtained at the end of step d) of the treatment process according to the invention is advantageously used as feedstock in a “second-generation” process for producing solvents and/or alcohols from lignocellulosic biomass.

The invention also relates to a process for producing solvents and/or alcohols from lignocellulosic biomass, comprising at least the following steps:
i) treating said lignocellulosic biomass by the treatment process according to the invention so as to obtain a treated lignocellulosic substrate;
ii) carrying out an enzymatic hydrolysis of the treated lignocellulosic substrate so as to obtain a hydrolyzate containing fermentable sugars;
iii) carrying out a fermentation of the hydrolyzate obtained from step ii) in order to obtain a fermentation must containing solvents and/or alcohols;
iv) separating the solvents and/or alcohols from the fermentation must.

Following the steam explosion under acidic conditions, the pretreated lignocellulosic substrate generally has a pH lower than that which is compatible with the medium for enzymatic hydrolysis. Thus, the lignocellulosic substrate treated according to step I) of the process for producing solvents and/or alcohols is subjected to a neutralization step to bring its pH to a value between 4 and 6.

For the neutralization step, an aqueous solution is used that contains a neutralizing agent which can be chosen from all weak or strong bases known to those skilled in the art. The term base denotes any chemical species which, when it is added to water, gives an aqueous solution with a pH of greater than 7. Preferably, the neutralizing agent is chosen from potassium hydroxide, sodium hydroxide, aqueous ammonia and lime. More preferably still, the neutralizing agent is chosen from potassium hydroxide and aqueous ammonia, alone or in combination with one another. Preferably, the neutralizing agent is used in aqueous solution, with a weight concentration of between 2% and 75%, and more preferably still between 20% and 70%.

Neutralization is carried out at a temperature between 15° C. and 95° C., and preferably between 20° C. and 70° C. In general, the temperature of the neutralization step is not precisely controlled and is simply governed by the heat given off by the acid-base neutralization reaction.

The neutralization step can be carried out continuously, in batch mode or in fed-batch mode.

It should be noted that an optional washing step can be carried out before or after the neutralization step, on all or part of the pretreated lignocellulosic substrate.

If a washing is applied, a liquid stream is brought into contact with the pretreated lignocellulosic substrate, then the liquid is separated from the solid. The washing step can be carried out by percolation, by successive mixing and liquid/solid separation operations, by washing on a belt filter or by any other technique known to those skilled in the art. The washing liquid used can be water or a process stream. The weight ratio between the washing liquid added and the liquid contained in the substrate to be washed is generally between 0.5 and 4. The washing step generates a sugary washing liquor containing a portion of the hemicelluloses solubilized during the pretreatment. This washing liquor can for example be used as a source of carbon for the production of biocatalysts (enzymes and/or microorganisms). The washing step is generally carried out at a temperature between 10° C. and 95° C.

### Step ii) of Enzymatic Hydrolysis

The pretreated lignocellulosic substrate, optionally neutralized and washed, is sent to the enzymatic hydrolysis step ii) of the process. The pretreated lignocellulosic substrate which is sent to the enzymatic hydrolysis step has a solids content generally of between 15% and 70% by weight. The objective of the enzymatic hydrolysis is to hydrolyze (depolymerize), by means of biocatalysts, the hemicelluloses and cellulose into fermentable sugars, preferably glucose.

The enzymatic hydrolysis step is carried out under mild conditions, at a temperature of the order of 40° C. and 55° C., preferably between 45° C. and 50° C. and at a pH of from 4.0 to 5.5, and more preferentially still between 4.8 and 5.2. The solids content of the enzymatic hydrolysis medium is between 5% and 45% by weight, preferably between 10% and 30% by weight. The enzymatic hydrolysis is carried out by means of enzymes produced by a microorganism. Natural or genetically modified microorganisms, such as the fungi of the genera Trichoderma, Aspergillus, Penicillium or Schizophyllum, or anaerobic bacteria of, for example, the genus Clostridium, produce a cocktail of enzymes containing in particular cellulases and hemicellulases, suitable for extensive hydrolysis of cellulose and hemicelluloses.

The enzymatic hydrolysis can be carried out continuously or in batch mode or in fed-batch mode, in one or more reactors. The residence time is between 12 hours and 200 hours and preferably between 24 hours and 120 hours and more preferentially still between 48 hours and 120 hours.

At the end of step ii), a hydrolyzate containing fermentable sugars is recovered from the bioreactor, which hydrolyzate is then treated in the fermentation step iii).

It should be noted that the hydrolyzate obtained can optionally undergo one or more treatment steps before the fermentation step. For example, these may be a return to the pH, a partial purification with a view to limiting the content of inhibitor compound for the fermentation microorganism, or an at least partial separation of the solid residues contained in the hydrolyzate.

Step iii) of Fermentation of the Hydrolyzate

According to step iii) of the process for producing solvents and/or alcohols, the optionally treated hydrolyzate is sent to the fermentation step enabling the conversion, by means of one or more microorganisms of different genera, of the fermentable sugars into solvent and/or alcohols of interest. The fermentaion methods are known to those skilled in the art.

The term “solvent” is intended to denote organic compounds other than alcohols, for example organic compounds having a ketone function such as acetone.

The term “alcohol” denotes in particular ethanol, propanol, isopropanol and butanol.

The natural or genetically modified microorganisms can be chosen, for example, from Saccharomyces cerevisiae, Schizosaccharomyces pombe, Saccharomyces uvarum, Saccharomyces diastaticus, Kluyveromyces fragilis, Candida shehatae, Pichia stipitis, Pachysolen tannophilus or the bacteria Zymomonas mobilis, Clostridium acetobutylicum, Escherichia coli.

In the context of the invention, the fermentation step makes it possible, for example, to produce ethanol alone or as a mixture with butanol, propanol, isopropanol and/or acetone. For example, the fermentation microorganism may be capable of producing an “ABE (acetone-butanol-ethanol)” mixture or else an “IBE (isopropanol-butanol-ethanol)” mixture.

Preferably, the microorganism chosen is a natural or genetically modified yeast of the genus Saccharomyces capable of producing ethanol.

At the end of step iii), a fermentation must, diluted in products of interest, is recovered.

According to one embodiment of the process, steps ii) and iii) are carried out at the same time, in at least one and the same bioreactor, so that the enzymatic hydrolysis and the fermentation are carried out simultaneously according to a process denoted by the term “Simultaneous Saccharification and Fermentation (SSF)”. When the hydrolysis step is merged with the fermentation step, the operating conditions, in particular temperature conditions, can be adapted to be compatible with the tolerances of the fermentation microorganism. For example, the temperature can be lowered between 28° C. and 45° C., and preferably between 30° C. and 35° C., when the fermentation is carried out with a yeast of the genus Saccharomyces. The pH is preferably adjusted between 5 and 5.5 in order to promote the performance of the yeasts.

The production unit carrying out the process according to the invention may comprise, in addition to the plants already described, units for the in situ production of enzymes and/or yeasts.

Step iv) of Separating the Solvents and/or Alcohols from the Fermentation Must

The process according to the invention can finally comprise a step of separating the product(s) of interest from the fermentation must, optionally preceded by a solid/liquid separation step in order to remove at least one fraction of the solid matter contained in the fermentation must.

Preferably the step of separating the product(s) of interest, for example ethanol, uses one or more distillations, which is technology well known to those skilled in the art.

The feedstock: lignocellulosic biomass

In accordance with the invention, the feedstock of the process may be a biomass alone or as a mixture. The amount of water contained in the crude feedstock is generally at least 10%, notably between 10% and 70% by weight.

The crude biomass is chosen from any type of biomass, preferably biomass of solid type, and in particular biomass of lignocellulosic type. Nonlimiting examples of biomass types concern, for example, farming residues (notably straw, corn cobs), forestry management residues, forestry management products, wood mill residues and dedicated crops, for example short-rotation coppice.

Preferably, the crude biomass, also referred to as native biomass, is lignocellulosic biomass. It essentially comprises three natural constituents present in variable amounts depending on its origin: cellulose, hemicellulose and lignin.

The lignocellulosic biomass feedstock is preferably used in its crude form, i.e. containing all of these three cellulose, hemicellulose and lignin constituents.

In one preferred embodiment of the invention, the lignocellulosic biomass is chosen from grassy biomass, farming residues such as straw waste, corn cobs, sugarcane bagasse, forestry management residues or wood mill residues such as wood chips or any other type of woody residues.

### The Impregnation Fluid

The optional fluid, injected for the impregnation, is an aqueous liquid solution optionally containing acid, at a temperature between 10° C. and 95° C. and at atmospheric pressure. The pH of this chemical solution is between 0.1 and 12.0, preferably between 0.1 and 7, preferably between 0.3 and 2. According to a preferred embodiment, the liquor used is an acid catalysis liquor, and the pH of the liquor is adjusted to between 0.1 and 4, notably between 0.3 and 2. Examples of acids that may be used include at least one acid chosen from sulfuric acid, hydrochloric acid, nitric acid and oxalic acid. Their content, in the aqueous phase, is preferably between 0.2% and 8% by weight.

Returning to the various FIGS. 2 to 8 one by one: All these figures are variations of a biomass pretreatment. It should be noted that they are described by providing an impregnation by an acid fluid then a steam explosion cooking, in order to illustrate the invention simply, but that the invention applies just as well to a pretreatment without impregnation, or with non-acidic impregnation, or else to cooking without steam explosion.

FIG. 2 represents the diagram of the pretreatment part of the biomass conversion process which can for example be applied to the plant from FIG. 1, with a known sequence of an (optional) impregnation by an impregnation fluid followed by cooking/steam explosion of the biomass, that the invention will modify: The (ground) biomass 5 enters a transfer zone comprising a compression screw feed device 6, which may optionally be washed by circulation of a washing fluid 7 of water type (discharged in the form of a used washing liquid 8), and which conveys the biomass into the impregnation reactor 9, which is also fed with impregnation fluid 4 via a preparation tank 3 fed with water 1 and with impregnation compound 2 (of acid or base type). As indicated above, there may be no impregnation. The impregnation may also be carried out differently, in batch mode (soaking of the biomass in a tank containing the impregnation fluid), or in continuous mode, by spraying for example, or by passage of the biomass through a stirred reactor.

It is thus possible to have a feed mode and an impregnation mode that are different, such as a conveyor belt conveying the biomass under a device for spraying impregnation fluid.

The impregnated biomass 10 then passes into a transfer zone comprising a compression feed screw 11, washed by washing fluid 12 (washing water), the used washing water 13 being discharged. Next, the biomass feeds a cooking reactor 14 in the presence of steam 15, then is extracted from the reactor in the form of pretreated biomass 16, in order to continue its process of conversion into sugars or alcohol.

FIG. 3 displays the first modification step by the invention of the process according to FIG. 2: The fluid 8 extracted from the feed device 6 of the impregnation reactor 9 may be water originating from the native biomass (or water added to the biomass before entering the feed device) and/or used washing water obtained from the washing water 7. This fluid 8 in fact consists of a liquid phase (water) and suspended solid particles (of biomass). According to the invention, it is conveyed to a solid/liquid separation device 30, at the outlet which a liquid phase/residue 31 and a solid residue 32 are recovered. This device may for example be a screening unit. These two residues will thus be able to be upgraded/reused separately in a very advantageous manner, as described below.

In an analogous manner, the fluid 13 extracted from the feed device 11 of the cooking reactor 14 also consists of a liquid phase (water) and suspended solid particles (of biomass). According to the invention, it is conveyed to a solid/liquid separation device 40, at the outlet which a liquid phase/residue 41 and a solid residue 42 are recovered. This device may for example also be a screening unit. These two residues will thus be able to be upgraded/reused separately in a very advantageous manner, as described below.

This separation and these upgradings/reuses of the solid and liquid residues may be carried out only on the feed device of the impregnation device/reactor 9 or only on the feed device of the cooking device/reactor 14, or on both devices, naturally.

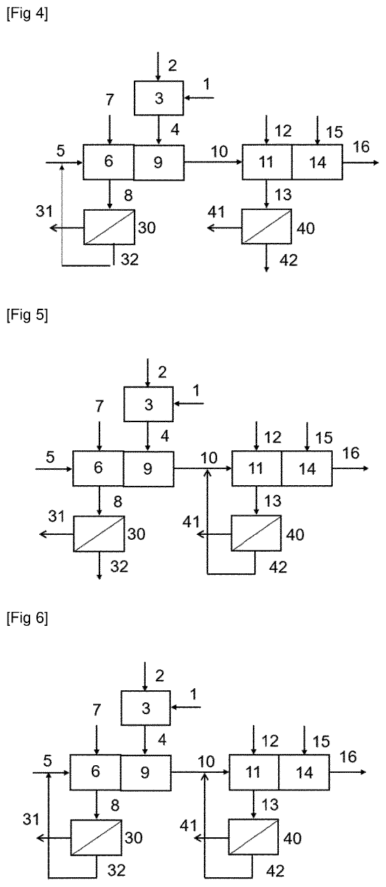

FIG. 4 illustrates an upgrading of the solid residue 32 (impregnation step), by reinjection of the residue 32 with the biomass feedstock 5 at the inlet of the feed device 6. In this scenario, the solid residue 32 has a composition that is similar/very similar, or even identical, to that of the biomass 5, but it may be in the form of particles of smaller size (when the feed device is of compression screw type, which has a mechanical action on the biomass.

The recycling of the solid residue 32 to the inlet of the impregnation reactor 9 with the ground biomass 5 makes it possible not to lose this solid stream, and to obtain a “clear” liquid residue 31 (which is free of visible suspended particles), which is also reusable. This solid recycling makes it possible to increase the yield of the process, since the residue 32 contains sugar polymers, to increase the lignin yield, since the residue 32 contains lignin, and to reduce the water consumption of the process, since the residue also contains water. The solid/liquid separation may be carried out on all or some of the stream 8 leaving the feed device 6, and this recycling of the solid residue 32 may be carried out on all or some of said solid residue.

FIG. 5 illustrates an upgrading of the solid residue 42 (cooking step), by reinjection of the residue 42 at the inlet of the feed device 12 of the cooking device/reactor 14. In this scenario, the solid residue 42 has a different composition to the residue 32, insofar as it was obtained from biomass impregnated by an impregnation liquid, notably which is highly acidic, and/or from washing waters of the feed device which may also contain a certain content of this fluid/acid.

The recycling of the solid residue 42 with the impregnated biomass 10 makes it possible not to lose this solid stream, and to obtain a clear liquid residue 41 (also referred to as pressate). This solid recycling makes it possible to increase the yield of the process, since the stream 42 contains sugar polymers, to increase the lignin yield, since the stream 42 contains lignin, and to reduce the consumption of impregnation liquid 2, since the residue 42 contains impregnation liquid. The solid/liquid separation may be carried out on all or some of the stream 13 leaving the feed device 11, and this recycling of the solid residue 42 may be carried out on all or some of said solid residue.

It is also possible to recycle all or some of the solid residue 42 with the ground biomass 5 at the impregnation inlet, but this recycling seems less advantageous, since a second impregnation of the biomass, which is already acidic, with the residue 42 is then carried out.

FIG. 6 represents both the reinjection of the solid residue 32 according to FIG. 4 and that of the solid residue 42 according to FIG. 5, and therefore illustrates an embodiment where the invention is applied twice in the biomass conversion process, with the same advantages as those described regarding FIGS. 4 and 5. Each of the reuses of the two solid residues may be carried out on the whole of each of the residues or on a portion only thereof. It is also possible to envisage combining all or some of the two solid residues in order to reuse them together in the process.

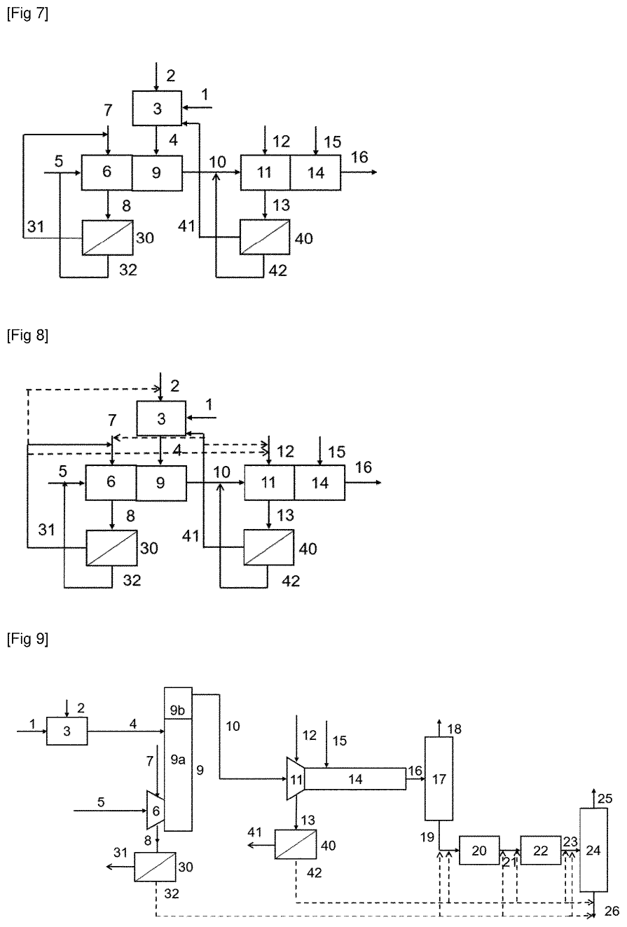

FIG. 7 represents the reinjection of the two solid residues 32 and 42 in accordance with FIG. 6, but also the reuse of the corresponding two liquid residues 31 and 41:
the liquid residue 31 is reinjected into the washing inlet of the feed device 6 of the impregnation device, it is essentially water.
the liquid residue 41 is reinjected into the impregnation liquor preparation tank. Specifically, this residue 41 contains a certain content of acid (if the example of an acid liquor continues to be taken), and re-injecting it into the tank 3 provides an advantageous makeup of water and acid.

The recycling of the liquid residues 31 and 41 enables the water consumption and consumption of impregnation liquid to be reduced. This recycling can be carried out on all or a portion of the liquid residues. Like for the solid residues, it is also possible to envisage combining them, at least in part, in order to recycle them together in the process.

FIG. 8 proposes variants, relative to FIG. 7, for reusing the liquid residues 31 and 41: the dotted arrows indicate various alternative or cumulative possibilities, for re-injecting them completely or partly into the biomass pretreatment process. Thus, the liquid residue 31 (the one upstream of the impregnation device) can also be reinjected, completely or partly, into the impregnation liquor preparation tank 3 as makeup water. It can also, completely or partly, be rejected as washing water 12 for the feed device 11 of the cooking device 14, or as washing water 7 for the feed device 6 of the impregnation device 9, if washing is provided for this device also. It can also, completely or partly, be added to the liquid residue 41 and follow its reinjection circuit. Conversely, the liquid residue 41 can itself also, completely or partly, be added to the liquid residue 31, before being jointly reused in the tank 3 for preparing the liquor 4 for example (these two variants not being represented in the figures).

It should also be noted that these liquid residues 31, 41, and notably at least the residue 41 which contains acid, may also be reused/re-injected directly in the impregnation device 9, if its acid content is, preferably, controlled upstream.

It should finally be noted that at least one of these liquid residues 31, 41 may also be reused in the biomass conversion process downstream of the pretreatment thereof, in a step subsequent to the cooking thereof which needs a supply of water and/or a supply of water having an acid pH.

FIG. 9 proposes a modification according to the invention of the plant described in FIG. 1: here, the two solid residues obtained from the feed devices of the impregnation reactor 9 and of the cooking reactor 14, or at least one of them, is at least partly reintroduced downstream of the biomass pretreatment. The dotted lines indicate the various alternative or cumulative options, for reintroducing these solid residues, completely or partly: they can therefore be reintroduced at the inlet of the hydrolysis reactor 20 or of the fermentation reactor 22 or even at the inlet of the separation equipment 24, notably via their own feed devices, which are conventional (for example, pipe(s) or endless screw system opening into an inlet made in the reactor). The solid residue thus mixes with the pretreated biomass stream 19, and/or with the hydrolyzate stream 21 and/or with the fermentation must 23.

A last option represented in FIG. 9 consists in directly introducing all or some of the solid residue 32 and/or 42 into the final residue 26 obtained from the separation of the fermentation must that can be upgraded as fuel. In this case, the amount of final residue 26, and therefore the amount of fuel that can be reused in the plant itself or outside the plant, are directly increased. This is another type of upgrading, which seeks not to increase the biomass conversion efficiency of the process, but to make the best use of the residues of the process.

### EXAMPLES

They relate to a process for pretreatment of lignocellulosic biomass with acid liquor impregnation then steam explosion, as described in FIG. 1.

The term “potential sugar (xylose, glucose)” used below defines edition of the various sugars, irrespective of their form: monomeric or polymeric sugar. Specifically, after pretreatment by cooking, a portion of the sugars remains in sugar polymer form (cellulose or hemicellulose for example), and a portion of the sugars is in the sugar monomer form (glucose or xylose for example). This measurement may be carried out using the standard ASTM E1758-01 (2020) “Standard Test Method for Determination of Carbohydrates in Biomass by High Performance Liquid Chromatography”. As defined in the standard, to express this amount of sugars in polymer form (cellulose for example), it is necessary to subtract the water of hydrolysis from this amount.

### Example 1

### Comparative

The biomass 5 is a wheat straw lignocellulosic biomass. Its composition is indicated in table 1 below:

TABLE 1

| | | |
|---|---|---|
| Cellulose | 34% | weight |
| Hemicellulose | 27% | weight |
| Lignin | 15% | weight |
| Water | 10% | weight |
| Others | 14% | weight |

After hydrolysis, the cellulose is converted into glucose or into glucose oligomers, and the hemicellulose is converted into xylose or into xylose oligomers.

642 kg/h of this 50 mm ground biomass 5 enters the process with a throughput of 200 kg/h of washing water 7. A first solid/liquid stream 8 of 203.8 kg/h leaves the process, in this stream is 1.3 kg/h of potential glucose and 1.1 kg/h of potential xylose. In the impregnation step (reactor 9), 1622.6 kg/h of water and 84.2 kg/h of sulfuric acid are added to the reactor from the preparation tank 3 and constitute the impregnation liquid 4.

At the inlet of the cooking reactor 14, the transfer zone 11 is fed with the impregnated biomass 10. The transfer zone 11 (compression screw) is washed with 4087.0 kg/h of water 12, a second solid/liquid stream 13 of 5099.9 kg/h leaves zone 11. In this stream 13 are 2.7 kg/h of potential glucose, 2.2 kg/h of potential xylose and 45.6 kg/h of acid. The cooking reactor 14 is heated by a steam stream 15 of 3471.1 kg/h. At the outlet of this reactor 14, the stream of pretreated biomass 16 of 4803.8 kg/h, comprising 237.8 kg/h of potential glucose and 155.2 kg/h of xylose leaves.

At the limits of the pretreatment, i.e. between the entry of the biomass 5 into the feed device 6 and its outlet from the cooking reactor 14, 98.1% by weight of potential glucose and 78.8% by weight of potential xylose have therefore been retained.

### Example 2

### in Accordance With the Invention

It is carried out in accordance with the variant of the invention represented in FIG. 4: the solid residue recovered is recycled to the inlet of the impregnation reactor.

The biomass feedstock is the same as that in example 1. The throughput is the same as for example 1:642 kg/h of this 50 mm ground biomass 5 enters the process with a throughput of 200 kg/h of washing water 7. A first solid/liquid stream 8 of 203.8 kg/h leaves the transfer zone 6 to the impregnator 9. This stream is separated in a separation tool 30 into a liquid stream/residue 31 of 192 kg/h and a solid stream/residue 32 of 11.8 kg/h. The separation tool 30 is a screening unit.

This solid residue 32 resulting from the solid/liquid separation is relatively wet (25% SC), and is composed of 1.3 kg/h of potential glucose and 1.1 kg/h of potential xylose. It is recycled with the ground biomass 5 at the inlet of the feed device 6. Surprisingly, the recycling of this solid residue 32 at the impregnation inlet does not give rise to accumulation in the feed device 6 despite its small particle size and despite the fact that the solid residue is first passed through the holes of the cage of the feed screw: it is entrained by the ground biomass 5 into the impregnation reactor 9. In the impregnation step, the same amounts of water and sulfuric acid are added to the tank 3 for preparing the impregnation liquid 4 as for example 1.

The rest of the process is identical to example 1. At the outlet of the cooking reactor 14, the stream of pretreated biomass 16 of 4815.5 kg/h, comprising 239.2 kg/h of potential glucose and 156.1 kg/h of potential xylose leaves.

At the limits of the pretreatment (same meaning of this term as in example 1), 98.6% by weight of potential glucose and 79.2% by weight of potential xylose, have therefore been retained, i.e. an increase in the yield of 1.0 point. Furthermore, the invention has made it possible to recycle a solid 32 which up to that point had no purpose.

### Example 3

### in Accordance With the Invention

It is carried out in accordance with the variant of the invention represented in FIG. 5: the solid residue recovered is recycled to the inlet of the cooking reactor. The same feedstock is treated here as in the two preceding examples.

642 kg/h of this 50 mm ground biomass 5 enters the process with a throughput of 200 kg/h of washing water 7. A first solid/liquid stream 13 of 203.8 kg/h leaves the process, by compression of the impregnated biomass in the feed screw 11. In this stream are 1.3 kg/h of potential glucose, and 1.1 kg/h of potential xylose. In the impregnation step, 1622.6 kg/h of water and 84.2 kg/h of sulfuric acid are added from the tank 3 for preparing the impregnation liquid 4.

The throughput is the same as for examples 1 and 2:642 kg/h of this 50 mm ground biomass enters the process (it enters into the impregnation reactor 9) with a throughput of 200 kg/h of washing water 7. A first solid/liquid stream 8 of 203.8 kg/h leaves the process. In this stream are 1.3 kg/h of potential glucose, and 1.1 kg/h of potential xylose. In the impregnation step (reactor 9), the same amounts of water are used as in examples 1 and 2, but only 84 kg/h of sulfuric acid (saving of 0.2 wt % compared to the preceding examples, are added to the tank 3 for preparing the impregnation liquid 4.

At the inlet of the cooking reactor 14, the transfer zone 11 is fed with the impregnated biomass 10. The transfer zone 11 is washed with 4087 kg/h of water 12, a second solid/liquid stream 13 of 5097.9 kg/h leaves the transfer zone 11 to the cooking reactor 14. This stream 13 is separated in a separation tool 40, here a screening unit, into a liquid stream/residue 41 of 5074.1 kg/h and a solid stream/residue 42 of 23.8 kg/h. This solid stream 42 resulting from the solid/liquid separation is relatively wet (26.1% SC), and is composed of 2.7 kg/h of potential glucose, 2.2 kg/h of potential xylose and 0.2 kg/h of sulfuric acid. It is recycled with the impregnated biomass 10. Surprisingly, the recycling of this residue 42 at the cooking inlet does not give rise to accumulation in the circuit in the compression screw feed device 11, despite its small particle size and despite the fact that the solid residue is first passed through the holes of the cage of the feed screw. It is entrained by the biomass 10 into the cooking reactor 14.

The cooking reactor 14 is heated by a steam stream 15 of 3471.1 kg/h. At the outlet of this reactor, the stream of pretreated biomass 16 of 4829.6 kg/h, comprising 240.5 kg/h of potential glucose and 157.4 kg/h of xylose leaves.

At the limits of the pretreatment (from the inlet into the feed device 6 to the outlet of the reactor 14), 99.2% by weight of potential glucose and 79.9% by weight of potential xylose, are therefore retained, i.e. an increase in the yield of 2.2 points. Furthermore, the solid 42 has been recycled, whereas up to that point it had no purpose, and the acid consumption has been reduced by 0.2 wt %.

Table 2 below indicates the compositions of the ground biomass 5 and of the solid residue 32 recovered from the feed device of the impregnation reactor 9.

TABLE 2

| | | | Solid residue 32 |
|---|---|---|---|
| | | | (impregnation |
| | | Biomass 5 | inlet) |
| Cellulose | wt % | 34 | 10.2 |
| Hemicellulose | wt % | 27 | 8.1 |
| Lignin | wt % | 15 | 4.5 |
| Water | wt % | 10 | 75.0 |
| Others | wt % | 14 | 2.2 |

It is seen that the residue 32 has a composition very similar to that of the starting biomass, with a significantly higher water content.

Table 3 below indicates once more the composition of the ground biomass 5 and that of the solid residue 42 recovered from the feed device of the cooking reactor 14:

TABLE 3

| | | | Solid residue |
|---|---|---|---|
| | | | 42 (cooking |
| | | Biomass 5 | inlet) |
| Cellulose | wt % | 34 | 10.1 |
| Hemicellulose | wt % | 27 | 8.0 |
| Lignin | wt % | 15 | 4.5 |
| Water | wt % | 10 | 73.9 |
| Sulfuric acid | wt % | 0 | 0.7 |
| Others | wt % | 14 | 2.9 |

It is seen that the residue 42 has a composition similar to that of the starting biomass, with a significantly higher water content, and a content of sulfuric acid in addition, which justifies the advantage of recycling it.

### Example 4

### in Accordance With the Invention

It is carried out in accordance with the variant of the invention represented in FIG. 6: here both the solid residue 32 recovered is recycled to the inlet of the impregnation reactor 9 and the solid residue 42 recovered is recycled to the inlet of the cooking reactor 14. The same feedstock is treated here as in the preceding examples.

The throughputs that are the results of the recyclings described in examples 2 and 3 apply to example 4: the yield is increased at the limits of the pretreatment by recycling these two solid residues, and the acid consumption is reduced.

At the limits of the pretreatment, 99.7% by weight of potential glucose and 80.3% by weight of potential xylose have been retained, i.e. an increase in yield of 3.2 points. Furthermore, two solids 32, 42 were recycled, which up to that point had no purpose. The combined crude throughput of these two residues is significant, since it represents 6% of the crude throughput of lignocellulosic biomass. Lastly, the acid consumption has been reduced by 0.2 wt %.

1. A process for treating a lignocellulosic biomass comprising a solids content of at most 90% by weight, said process comprising the use of at least one reactor (9, 14) for treating said biomass, said reactor (9, 14) being equipped with a biomass feed device (6, 11) which is equipped with a biomass inlet and a biomass outlet, said biomass outlet being in fluidic connection with an inlet of the reactor, characterized
in that a residue (8; 13) is extracted from the biomass, while it is passing through the feed device (6; 11) toward the reactor (9; 14), via an extraction outlet provided in said device, said residue, referred to as solid-liquid residue, being a mixture of solid and liquid,
in that said solid-liquid residue is separated into a solid residue (32; 42) and a liquid residue (31; 41),
and in that at least one portion of the solid residue (32; 42) is reintroduced into the same feed device, or into one of said feed devices in the case of a plurality of reactors.

2. The process as claimed in claim 1, characterized in that the or at least one of the feed devices (6; 11) is washed by circulation of a washing fluid (7; 12) between a washing inlet and a washing outlet of said feed device, the washing outlet preferably also being the extraction outlet for the solid-liquid residue.

3. The process as claimed in claim 1, characterized in that at least one portion of the liquid residue (31; 41) is reintroduced into the same feed device, or into one of said feed devices in the case of a plurality of reactors.

4. The process as claimed in claim 1, characterized in that said process comprises the use of several reactors (9; 14) for treating said biomass, in that at least one portion of the solid residue (32; 42) is reintroduced into one of the feed devices of said reactors, and in that at least one portion of the liquid residue (31; 41) is reintroduced into another of said feed devices.

5. The process as claimed in claim 1, characterized in that the or at least one of the feed devices (6; 11) creates a pressure increase between the biomass inlet of said device, this pressure increase generating a compression of the biomass leading to the extraction of the solid-liquid residue (8; 13).

6. The process as claimed in claim 1, characterized in that the or at least one of the feed devices (6; 11) is a feed screw, which is notably at least partly conical, comprising a cowling equipped with a cage equipped with openings which allow the extraction of the solid-liquid residue (8; 13) from the biomass and optionally the circulation of a washing fluid (7; 12).

7. The process as claimed in claim 1, characterized in that said solid-liquid residue (8; 13) is separated into a solid residue (32; 42) and a liquid residue (31; 41) by at least one separation device (30; 40) chosen from a centrifugation device, a draining or pressing device, or a screening unit.

8. The process as claimed in claim 1, characterized in that it comprises a step of impregnating the biomass with an impregnation liquor (4) containing a chemical catalyst, said step being carried out by introducing the biomass into the or one of the impregnation reactors (9) through its/their feed device (6).

9. The process as claimed in claim 1, characterized in that it comprises a step of treating the biomass by cooking or steam explosion, said step being carried out by introducing the biomass into the or one of the cooking reactors (14) through its/their feed device (11).

10. The process as claimed in claim 1, characterized in that it comprises at least one step of treating the biomass by enzymatic hydrolysis, said step being subsequent to the cooking or steam explosion thereof, in an enzymatic hydrolysis reactor (20) equipped with its feed device.

11. The process as claimed in claim 1, characterized in that it comprises at least one step of treating the biomass by fermentation, said step being subsequent to or concomitant with the enzymatic hydrolysis step, in a fermentation reactor (22) equipped with its feed device.

12. The process as claimed in claim 8, characterized in that a residue (8; 13) is extracted from the biomass, while it is passing through the feed device (6;11) toward the impregnation reactor (9) and/or toward the cooking reactor (14), via an extraction outlet provided in said device, said residue, referred to as solid-liquid residue, being a mixture of solid and liquid,
in that said solid-liquid residue is separated into a solid residue (32; 42) and a liquid residue (31; 41),
and in that at least one portion of the solid residue (32; 42) is reintroduced into the same feed device (6; 11) or into at least one of the feed devices of the enzymatic hydrolysis reactor (20) and/or of the fermentation reactor (22) and/or of the separation reactor (24).

13. The process as claimed in claim 9, characterized in that at least one portion of the solid residue (32; 42) is reintroduced into the feed device of the impregnation reactor (9) or into the feed device of the cooking reactor (14), and in that the liquid residue (31; 41) is reused for the preparation of the impregnation liquor.

14. The process as claimed in claim 10, characterized in that at least one portion of the solid residue (32; 42) is reintroduced into the feed device of the enzymatic hydrolysis reactor (20), and in that the liquid residue (31; 41) is reused for the preparation of the impregnation liquor.

15. The process as claimed in claim 1, said process comprising the following steps:
a) preparing an impregnation liquor (4) containing a chemical catalyst (2) intended for the impregnation of the biomass (5), the catalyst being chosen from an acid catalyst, a basic catalyst and an oxidizing catalyst, and preferably an acid catalyst, in a preparation zone (3),
b) introducing the biomass (5) into an impregnation reactor (9) by means of a first feed device (6), said first feed device being washed by circulation of a first washing fluid (7) between a washing inlet and a washing outlet of said device (6),
c) introducing the liquor into the impregnation reactor (9) via a first liquor (4) inlet of the reactor,
d) transferring the impregnated then drained biomass (10) from an impregnation reactor outlet to an inlet of a cooking pretreatment reactor (14) via at least one second feed device (11), said second feed device being washed by circulation of a second washing fluid (12) between a washing inlet and a washing outlet of said feed device,
e) pretreating said biomass (10) in said reactor (14) by cooking or steam explosion,
f) extracting from the biomass a solid-liquid residue (8; 13) passing through at least one of the two feed devices (6; 11), then separating said/each of the solid-liquid residues into a solid residue (32; 42, and a liquid residue (31; 41) with reintroduction of at least one portion of the solid residue as biomass makeup into at least one of the feed devices (6; 11), and reintroduction of at least one portion of the liquid residue (31; 41) as washing fluid makeup into at least one of the feed devices (6; 11, or as makeup for the impregnation liquor in the liquor preparation zone (3) or in the impregnation reactor (9).
